SEQUENCE LISTING (33 sequences)

SEQ ID NO: 1 - DNA, 46 bp, synthetic construct
  source 1..46: mol_type = other DNA
cagccagccgcagcacgttcgctcataggagatatggtagagccgc

SEQ ID NO: 2 - DNA, 51 bp, synthetic construct
  source 1..51: mol_type = other DNA
gagagaatttgtaccacctcccaccgggcacatagcagtcctagggacagt

SEQ ID NO: 3 - DNA, 22 bp, synthetic construct
  source 1..22: mol_type = other DNA
ggcttggctctgctaacacgtt

SEQ ID NO: 4 - DNA, 22 bp, synthetic construct
  source 1..22: mol_type = other DNA
ggacgtttgtaatgtccgctcc

SEQ ID NO: 5 - DNA, 17 bp, synthetic construct
  source 1..17: mol_type = other DNA
ctgcatacgacgtgtct

SEQ ID NO: 6 - DNA, 20 bp, synthetic construct
  source 1..20: mol_type = other DNA
accatctatgactgtacgcc

SEQ ID NO: 7 - DNA, 41 bp, synthetic construct
  source 1..41: mol_type = other DNA
tctggcccagttcctaggtagtccagacgaattcgtggtgg

SEQ ID NO: 8 - DNA, 40 bp, synthetic construct
  source 1..40: mol_type = other DNA
agacggcatcatatgggttgcacgggtgccaatgtgatct

SEQ ID NO: 9 - DNA, 19 bp, synthetic construct
  source 1..19: mol_type = other DNA
tggctactaccgaagagct

SEQ ID NO: 10 - DNA, 20 bp, synthetic construct
  source 1..20: mol_type = other DNA
tgcagcattgttagcaggat

SEQ ID NO: 11 - DNA, 25 bp, synthetic construct
  source 1..25: mol_type = other DNA
ggactgagatctttcattttaccgt

SEQ ID NO: 12 - DNA, 21 bp, synthetic construct
  source 1..21: mol_type = other DNA
actgagggagccttgaataca

SEQ ID NO: 13 - DNA, 42 bp, synthetic construct
  source 1..42: mol_type = other DNA
accacgaaagcaagaaaaagaagttcgtttcggaagagacag

SEQ ID NO: 14 - DNA, 44 bp, synthetic construct
  source 1..44: mol_type = other DNA
ttgctagttacactagccatccttaggttttacaagactcacgt

SEQ ID NO: 15 - DNA, 23 bp, synthetic construct
  source 1..23: mol_type = other DNA
tgagtacgaacttatgtactcat

SEQ ID NO: 16 - DNA, 22 bp, synthetic construct
  source 1..22: mol_type = other DNA
ttcagatttttaacacgagagt

SEQ ID NO: 17 - DNA, 18 bp, synthetic construct
  source 1..18: mol_type = other DNA
cgctattaactattaacg

SEQ ID NO: 18 - DNA, 19 bp, synthetic construct
  source 1..19: mol_type = other DNA
gcgcttcgattgtgtgcgt

SEQ ID NO: 19 - DNA, 19 bp, synthetic construct
  source 1..19: mol_type = other DNA
cacattggcacccgcaatc

SEQ ID NO: 20 - DNA, 20 bp, synthetic construct
  source 1..20: mol_type = other DNA
gaggaacgagaagaggcttg

SEQ ID NO: 21 - DNA, 25 bp, synthetic construct
  source 1..25: mol_type = other DNA
  misc_feature 1: note = 5' 6-FAM
  misc_feature 25: note = 3' BHQ-1
acttcctcaaggaacaacattgcca

SEQ ID NO: 22 - DNA, 26 bp, synthetic construct
  source 1..26: mol_type = other DNA
acaggtacgttaatagttaatagcgt

SEQ ID NO: 23 - DNA, 22 bp, synthetic construct
  source 1..22: mol_type = other DNA
atattgcagcagtacgcacaca

SEQ ID NO: 24 - DNA, 26 bp, synthetic construct
  source 1..26: mol_type = other DNA
  misc_feature 1: note = 5' 6-FAM
  misc_feature 26: note = double-quenched internal ZEN with 3' IBFQ
acactagccatccttactgcgcttcg

SEQ ID NO: 25 - DNA, 46 bp, synthetic construct
  source 1..46: mol_type = other DNA
gccccacatgaaaagaatgttctttgttgactcaaaagaacaacgc

SEQ ID NO: 26 - DNA, 41 bp, synthetic construct
  source 1..41: mol_type = other DNA
aatcacccacccactgatactatcttctgttgccacacttg

SEQ ID NO: 27 - DNA, 22 bp, synthetic construct
  source 1..22: mol_type = other DNA
cattctacagaagcatgagatg

SEQ ID NO: 28 - DNA, 19 bp, synthetic construct
  source 1..19: mol_type = other DNA
aatggtttgaaggccctat

SEQ ID NO: 29 - DNA, 23 bp, synthetic construct
  source 1..23: mol_type = other DNA
gtgtattgggcgtcttgaatagg

SEQ ID NO: 30 - DNA, 21 bp, synthetic construct
  source 1..21: mol_type = other DNA
acgcagacagatctgtacaca

SEQ ID NO: 31 - DNA, 18 bp, synthetic construct
  source 1..18: mol_type = other DNA
  variation 14: note = can be replaced by thymine (T)
atttattcgactgccgcc

SEQ ID NO: 32 - DNA, 17 bp, synthetic construct
  source 1..17: mol_type = other DNA
  variation 9: note = can be replaced by thymine (T)
atgttgcacctgcaaga

SEQ ID NO: 33 - DNA, 18 bp, synthetic construct
  source 1..18: mol_type = other DNA
  misc_feature 1: note = 5' 6-FAM
  misc_feature 18: note = 3' BHQ-1
  variation 7: note = can be replaced by guanine (G)
  variation 4: note = can be replaced by guanine (G)
tgcaatagggtttgcgcc

(19) United States
(12) Patent Application Publication
(10) Pub. No.: US 2025/0297302 A1
(43) Pub. Date: Sep. 25, 2025

(54) UNIVERSAL FLUORESCENT NANOPROBES FOR ONE-STEP ISOTHERMAL NUCLEIC ACID AMPLIFICATION ASSAY

(71) Applicant: THE HONG KONG POLYTECHNIC UNIVERSITY, Hong Kong (CN)

(72) Inventors: Ming Hung Thomas LEE, Hong Kong (CN); Shea Ping YIP, Hong Kong (CN); Shiyao WANG, Hong Kong (CN); Ailin QIN

(21) Appl. No.: 18/716,607

(22) PCT Filed: Dec. 9, 2022
(86) PCT No.: PCT/CN2022/137841

§ 371 Date: Jun. 5, 2024

Related U.S. Application Data
(60) Provisional application No. 63/265,363, filed on Dec. 14, 2021.

(51) Int. Cl. C12Q 1/6844

(52) U.S. Cl. CPC C12Q 1/6844

(57) ABSTRACT

The present invention provides a universal fluorescent probing system and method for result readout of loop-mediated isothermal amplification (LAMP) or reverse transcription-loop-mediated isothermal amplification (RT-LAMP) in a closed-tube manner using at least three different types of universal quantum dot probes, including sulfonate-, carboxyl-, and amine-modified quantum dots (QDs). Detection mechanism of the present system and method relies on a co-precipitation of the modified QDs with magnesium pyrophosphate crystals (one of the positive LAMP/RT-LAMP reaction products) formed during the reaction. The present system and method are advantageous over the existing technologies in terms of simple preparation, low cost, as well as excellent universality, specificity and sensitivity.

### CROSS-REFERENCE TO RELATED APPLICATION

This application claims priority from the U.S. provisional patent application Ser. No. 63/265,363 filed Dec. 14, 2021, and the disclosure of which is incorporated herein by reference in its entirety.

### REFERENCE TO SEQUENCE DISCLOSURE

A sequence listing file with a file name “P23893PCT00_Sequence_Listing.xml” in ST.26 XML file format having a file size of 34 KB created on Dec. 8, 2022 is incorporated herein by reference in its entirety.

### TECHNICAL FIELD

The present invention relates to universal fluorescent nanoprobes and method of using thereof for one-step isothermal nucleic acid amplification assay, in particular, to a plurality of universal quantum dot probes for use in a loop-mediated isothermal amplification (LAMP) reaction or reverse transcription-loop-mediated isothermal amplification (RT-LAMP) in a closed-tube manner.

### BACKGROUND

Nucleic acid testing has become a very powerful tool for medical diagnostics, food safety monitoring, environmental surveillance, and many other applications. The gold standard tests for deoxyribonucleic acid (DNA) and ribonucleic acid (RNA) targets are real-time polymerase chain reaction (also termed as quantitative PCR, qPCR) and real-time reverse transcription-polymerase chain reaction (RT-qPCR or qRT-PCR), respectively. PCR is mainly performed in clinical, medical, and central laboratories using bulky and costly thermocycling equipment.

There has been a huge demand for decentralized nucleic acid testing, especially since the COVID-19 pandemic from 2019. Isothermal nucleic acid amplification techniques are excellent candidates in view of their simple temperature control and fast amplification [Zhao et al., Chem. Rev., 115, 12491-12545 (2015)]. A number of isothermal amplification techniques have been developed, including nucleic acid sequence-based amplification (NASBA) [Compton, Nature, 350, 91-92 (1991)], strand displacement amplification (SDA) [Walker et al., Nucleic Acids Res., 20, 1691-1696 (1992)], rolling circle amplification (RCA) [Fire et al., Proc. Natl. Acad. Sci. U.S.A., 92, 4641-4645 (1995)], loop-mediated isothermal amplification (LAMP) [Notomi et al., Nucleic Acids Res., 28, e63 (2000)], exponential amplification reaction (EXPAR) [van Ness et al., Proc. Natl. Acad. Sci. U.S.A., 100, 4504-4509 (2003)], helicase-dependent amplification (HDA) [Vincent et al., EMBO Rep., 5, 795-800 (2004)], and recombinase polymerase amplification (RPA) [Piepenburg et al., PLoS Biol., 4, e204 (2006)]. Among them, LAMP is particularly attractive due to the high thermal stability of lyophilized reagents for room temperature storage [Chen et al., Milit. Med., 181, 227-231 (2016)] and high tolerance to unpurified samples for direct amplification [Kaneko et al., J. Biochem. Biophys. Methods, 70, 499-501 (2007); Francois et al., FEMS Immunol. Med. Microbiol., 62, 41-48 (2011)].

The ability to monitor the progress of the amplification reaction in real-time, typically by incorporating organic fluorophore into the reaction mixture, eliminates additional detection time and enables quantification over a wide dynamic range. The main drawback of this approach is that organic fluorophores have poor photostability, therefore, they must be stored and handled in darkness with extreme caution. Otherwise, the accuracy of the assay result would be significantly affected.

Fluorescent semiconductor nanocrystals (quantum dots; QDs) have been proved to be promising substitutes for organic fluorophores by virtue of their superior photostability and brightness [Resch-Genger et al., Nat. Methods, 5, 763-775 (2008)]. Efforts have been made to couple QD detection probes with isothermal nucleic acid amplification [Bakalova et al., J. Am. Chem. Soc., 127, 11328-11335 (2005); Zhang et al., Anal. Chem., 84, 224-231 (2012); Hu et al., Chem. Sci., 9, 4258-4267 (2018); Wang et al., ACS Appl. Mater. Interfaces, 10, 28290-28300 (2018)]. However, due to the postamplification open-tube addition of QD detection probes, these platforms were susceptible to carryover contamination. To date, only a few PCR-based closed-tube platforms (nucleic acid amplification coupled with QD detection probes) were reported [Kuang et al., Biosens. Bioelectron., 26, 2495-2499 (2011); He et al., Biomaterials, 32, 5471-5477 (2011); Cui et al., Nano Biomed. Eng., 2, 45-55 (2010)]. Almost all conventional QDs are modified with target-specific oligonucleotide sequence (complementary to target/amplified sequence), the preparation of which is costly (oligonucleotide with attachment functional group). It should be noted that different oligonucleotide-modified QD probes are needed for different targets (poor universality).

A need therefore exists for an improved universal fluorescent probing system without oligonucleotide attachment for a closed-tube monitoring of isothermal nucleic acid amplification reactions that at least diminishes or eliminates the disadvantages and problems described above.

### SUMMARY OF INVENTION

Accordingly, a first aspect of the present invention provides a universal fluorescent probing system for one-step isothermal nucleic acid amplification assay, including a plurality of functionalized fluorescent nanoprobes capable of forming coprecipitates in the presence of positive isothermal amplification reaction products along with a target sequence amplification.

In certain embodiments, the plurality of functionalized fluorescent nanoprobes includes one or more semiconductor quantum dots (QDs) with surface modification(s).

In certain embodiments, the one or more semiconductor QDs include cadmium selenide sulfide/zinc sulfide (CdSeS/ZnS core/shell) quantum dots.

In certain embodiments, the one or more semiconductor QDs are surface modified by one of 2-mercaptoethanesulfonate, mercaptoacetic acid, and cysteamine to result in sulfonate-QDs, carboxyl-QDs, and amine-QDs, respectively.

In certain embodiments, the positive isothermal amplification reaction products with which the plurality of functionalized fluorescent nanoprobes is capable of forming coprecipitates include magnesium pyrophosphate (Mg₂P₂O₇) crystals.

In certain embodiments, sulfonate-QDs or carboxyl-QDs form coprecipitates via complexation between Mg²⁺ and P₂O₇⁴⁻ of the Mg₂P₂O₇ crystals with the sulfonate or carboxyl group of the sulfonate-QDs and carboxyl-QDs in the course of the isothermal amplification reaction.

In certain embodiments, amine-QDs form coprecipitates via electrostatic attraction between P₂O₇⁴⁻ of the Mg₂P₂O₇ crystals and amine group of the amine-QDs in the course of the isothermal amplification reaction.

In certain embodiments, the isothermal amplification reaction includes loop-mediated isothermal amplification (LAMP) and reverse transcription-loop-mediated isothermal amplification (RT-LAMP) reactions.

In certain embodiments, the LAMP reaction is performed at about 65° C. for 1 hour or less.

A second aspect of the present invention provides a method for closed-tube detection of target sequence in a sample based on an isothermal amplification reaction, the method comprising:
providing the plurality of functionalized fluorescent nanoprobes of the universal fluorescence probing system in the first aspect or various embodiments of the present disclosure to an isothermal amplification reaction mixture containing the sample; incubating the plurality of the functionalized fluorescent nanoprobes with the isothermal amplification reaction mixture containing the sample at an isothermal amplification temperature for a time interval;
analyzing the isothermal amplification reaction mixture during or after said incubating by a fluorescence reader (supernatant portion of the reaction mixture); and determining the presence of the target sequence in the sample by detecting any fluorescence precipitates formed in the isothermal amplification reaction mixture during or after said incubating;
otherwise, detecting a fluorescence dispersion in the isothermal amplification reaction mixture after said incubating for verifying the absence of the target sequence in the sample.

In certain embodiments, the isothermal amplification reaction mixture includes isothermal amplification reagents capable of forming Mg₂P₂O₇ crystals when positive amplicons of the target sequence are generated by either LAMP or RT-LAMP reaction.

In certain embodiments, an analyte of the sample includes one or more types of nucleic acids directly obtained or isolated from a test subject, object, or biological sample.

In certain embodiments, the isothermal amplification temperature is about 65° C., and the time interval for said isothermal amplification is about 1 hour or less.

In certain embodiments, the LAMP or RT-LAMP reaction can be carried out in any device with uniform heating and cooling capabilities and a time control mechanism.

A third aspect of the present invention provides a method of fabricating the universal fluorescence probing system for an isothermal amplification reaction, the method includes:
selecting a semiconductor quantum dot;
modifying surface of said semiconductor quantum dot with one or more functional groups such that the one or more functional groups are capable of interacting with Mg²⁺ and/or P₂O₇⁴⁻ to form coprecipitates.

Other aspects of the present invention include a kit for detecting the presence of a target sequence in a sample comprising any one or more of the functionalized fluorescent nanoprobes as described in any of the foregoing aspects or according to various embodiments of the present invention. The kit may also include reaction components for LAMP or RT-LAMP reaction including, but not limited to, isothermal amplification buffer, deoxynucleotides, enzymes, betaine, LAMP primers with respect to the target sequence, stabilizers or additives to facilitate the formation of coprecipitates, a control sample, and solvent or solution for dissolving any reaction components. The kit may also include a portable device or platform for performing LAMP reaction and assessment by the functionalized fluorescent nanoprobes, such as a uniform heating and cooling plate with temperature and time control mechanisms for holding the LAMP reaction samples/mixtures and performing the LAMP reaction.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1 shows dispersion and precipitation behavior of sulfonate-, carboxyl-, and amine-modified quantum dots (QDs) under simulated LAMP conditions. Fluorescence photographs (365 nm ultraviolet excitation) of simulated negative LAMP samples (“Mg²⁺”; comprising 1× isothermal amplification buffer, 2 mM MgCl₂, and QDs) and simulated positive LAMP samples (“Mg₂P₂O₇”; comprising 1×isothermal amplification buffer, 2 mM MgCl₂, 1.4 mM K₄P₂O₇, and QDs). 1×isothermal amplification buffer contained 20 mM Tris-HCl, 10 mM (NH₄)₂SO₄, 50 mM KCl, 2 mM MgSO₄, and 0.1% Tween 20 (pH 8.8). The samples were incubated at 65° C. for 1 h.

FIG. 2 shows a schematic illustration of the three types of QD probes for closed-tube LAMP assay. For a negative LAMP sample, QD probes remain dispersed, thus a fluorescent dispersion is observed. For a positive LAMP sample, magnesium pyrophosphate (Mg₂P₂O₇) crystals are produced along with target sequence amplification. QDs are coprecipitated with Mg₂P₂O₇ crystals, thus a fluorescent precipitate is observed.

FIG. 3 shows LAMP detection with sulfonate-, carboxyl-, and amine-modified QDs: (a) Fluorescence photographs of the negative samples (“−ve”) without the specific template and positive samples (“+ve”) with the specific template (10⁵ copies of lambda DNA) subjected to LAMP (65° C. for 1 h); (b) Agarose gel electrophoresis results of the LAMP reaction products. Lane M: DNA ladder; lanes 1 and 2: controls without QDs; lanes 3 and 4: samples with QDs; lanes 1 and 3: without the specific template; and lanes 2 and 4: with the specific template.

FIG. 4 shows specificity (a, c, and e) and sensitivity (b, d, and f) of the closed-tube LAMP assays with sulfonate-, carboxyl-, and amine-modified QDs. (a, c, and e) Fluorescence photographs show the amplification results of four different template combinations of lambda DNA (specific template) and pBR322 DNA (nonspecific template): (from left to right) sample without template; sample with lambda DNA; sample with pBR322 DNA; and sample with lambda DNA and pBR322 DNA. The copy number of both templates was 10⁵. (b, d, and f) Fluorescence photographs showing the amplification results of different copy numbers of lambda DNA target (0-10⁵). (g) Plots of fluorescence intensity (supernatant) versus LAMP reaction time for the samples (with amine-modified QDs) containing 10³ or 10⁵ copies of lambda DNA.

FIG. 5 (a, c, and e) shows agarose gel electrophoresis results of the LAMP reaction products in the specificity tests. Lane M: DNA ladder; lanes 1-4: controls without QDs; lanes 5-8: samples with QDs; lanes 1 and 5: without any template; lanes 2 and 6: with specific template (10⁵ copies of lambda DNA); lanes 3 and 7: with nonspecific template (10⁵ copies of pBR322 DNA); and lanes 4 and 8: with both specific and nonspecific templates (10⁵ copies each). (b, d, and f) Agarose gel electrophoresis results of the LAMP reaction products in the sensitivity tests. Lane M: DNA ladder; lanes 1-6: controls without QDs; lanes 7-12: samples with QDs; lanes 1-6 as well as lanes 7-12: 0, 10, 10², 10³, 10⁴, and 10⁵ copies of lambda DNA.

FIG. 6 shows limit of detection (LOD) determination of the LAMP assays with sulfonate-, carboxyl-, and amine-modified QD probes. Fluorescence photographs show the amplification results of different copy numbers (0-1,000) of lambda DNA after incubation at 65° C. for 1 h.

FIG. 7A shows (Top) Sensitivity of the amine-QD-based one-step RT-LAMP assay for severe acute respiratory syndrome coronavirus 2 (SARS-CoV-2) RNA (nucleocapsid (N) gene and envelope (E) gene) detection. Fluorescence photographs showing the results of samples with different copy numbers of SARS-CoV-2 (0-100; triplicate for each; only one set of the triplicates is shown). (Bottom) Plots of fluorescence intensity versus cycle number for different copy numbers (0-100; triplicate for each) of SARS-CoV-2 in a real-time reverse transcription-polymerase chain reaction (RT-qPCR).

FIG. 7B shows agarose gel electrophoresis results of the sensitivity test of the amine-QD-based one-step RT-LAMP assay for SARS-CoV-2 RNA (N gene and E gene) detection in FIG. 7A. Lane M: DNA ladder; lanes 1-4: N gene; lanes 5-8: E gene; lanes 1 and 5: 0 copies of SARS-CoV-2 RNA; lanes 2 and 6: 1 copy of SARS-CoV-2 RNA; lanes 3 and 7: 10 copies of SARS-CoV-2 RNA; and lanes 4 and 8: 100 copies of SARS-CoV-2 RNA.

FIG. 7C shows specificity of the amine-QD-based one-step RT-LAMP assay for SARS-CoV-2 RNA (E gene) detection. Fluorescence photographs showing the results of samples with different templates (note: only SARS-CoV-2 was the specific template). NTC: no template control; BPE: Bordetella pertussis DNA; HIN: Haemophilus influenzae DNA; INF-AH1: influenza A H1 RNA; MERS: Middle East respiratory syndrome coronavirus RNA; MTU: Mycobacterium tuberculosis DNA; MPN: Mycoplasma pneumoniae DNA; PAR-1: parainfluenza 1 RNA; PAR-4A: parainfluenza 4A RNA; RHI: rhinovirus RNA; SARS (2003): SARS (2003) coronavirus RNA; SPN: Streptococcus pneumoniae DNA; and SARS-CoV-2 & all others: SARS-CoV-2 plus 11 nonspecific templates. The copy numbers of SARS-CoV-2 RNA and the nonspecific templates were 10² and 10³, respectively. The samples were incubated at 65° C. for 35 min.

FIG. 8 shows an avian influenza virus (H9 subtype) RNA detection from live chicken swab samples: (a) Schematic illustration of the sample preparation steps; (b) Fluorescence photograph showing the amine-QD-based one-step RT-LAMP results of serially diluted (10⁻³-10⁻⁶) H9 negative “−” and positive “+” samples; (c) Plots of fluorescence intensity versus cycle number for different dilutions (10⁻³-10⁻⁶; duplicate for each) of H9 negative and positive samples in RT-qPCR.

FIG. 9 shows agarose gel electrophoresis results of the amine-QD-based one-step RT-LAMP assay for serially diluted (10⁻³-10⁻⁶) H9 negative and positive samples in FIG. 8b, where Lane M: DNA ladder; lanes 1, 3, 5, and 7: negative; lanes 2, 4, 6, and 8: positive; lanes 1 and 2: 10⁻³ dilution; lanes 3 and 4: 10⁻⁴ dilution; lanes 5 and 6: 10⁻⁵ dilution; and lanes 7 and 8: 10⁻⁶ dilution

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The present disclosure provides a closed-tube detection of LAMP using three types of QD probes functionalized with sodium 2-mercaptoethanesulfonate (sulfonate-QDs), and mercaptoacetic acid (carboxyl-QDs), and cysteamine (amine-QDs), respectively.

Since one of the features of LAMP is the generation of Mg₂P₂O₇ crystals in a positive sample (presence of target sequence), which can be monitored by turbidity measurement. However, this turbidimetric assessment (normally by naked eye) is quite difficult to justify the turbidity readout, e.g., light intensity may affect the judgement of turbidity by naked eye observation.

Therefore, the three surface-modified (expression such as “modified”, “functionalized” or “surface-functionalized” used herein before quantum dots or “QDs” may also refer to the same meaning) QDs, i.e., sulfonate-QDs, carboxyl-QDs, and amine-QDs, according to certain embodiments of the present invention have been proposed in combination with LAMP reaction to enable a non-turbidimetric, fluorometric assessment for the presence of a target sequence in a sample without conjugation or modification of the QDs with target-specific oligonucleotide sequence which is usually complementary to the target sequence or amplicons containing the target sequence. These modified QDs in the present invention coprecipitate with magnesium pyrophosphate crystals, which is an indicator of positive LAMP samples (with target sequence). These modified QDs are also sensitive to the positive LAMP samples even in a low copy number of nucleotides with the target sequence (as evident by certain examples described hereinafter).

The modified QDs are preferably in nanoparticle form with surface modification by one or more functional groups capable of interacting with the magnesium pyrophosphate crystals to form coprecipitates.

In addition to the modified QDs according to certain embodiments, the present system also includes other amplification reaction components such as isothermal amplification buffer, deoxynucleoside triphosphates, enzyme(s), and betaine.

The present invention also provides a nucleic acid amplification detection method including: obtaining an analyte from a sample or directly from a test subject or object, where the analyte contains one or more types of nucleic acid to be amplified; designing specific primers or utilizing known specific primers based on a target nucleic acid sequence to be determined; mixing the analyte, the primers, the functional fluorescent nanoprobes or nanoparticles according to certain embodiments, and the other amplification reaction components used in the magnesium pyrophosphate crystal-producing nucleic acid amplification reaction to obtain a reaction mixture; incubating the reaction mixture under certain amplification conditions; and detecting a change in the fluorescence of the reaction mixture supernatant during or after the amplification reaction, wherein a change in the fluorescence of the supernatant indicates a positive sample (presence of the target nucleic acid sequence).

In certain embodiments, the change in the fluorescence of the amplification reaction supernatant is compared with that of a no-template control (NTC).

The amplification conditions according to certain embodiments include reaction temperature of about 65° C. and reaction time of about an hour or less, which can be varied subject to the concentration of different components including the copy number of the target nucleic acid samples to be amplified, pH of the reaction mixture, concentration of compounds responsible for forming the magnesium pyrophosphate crystal during the nucleic acid amplification, etc.

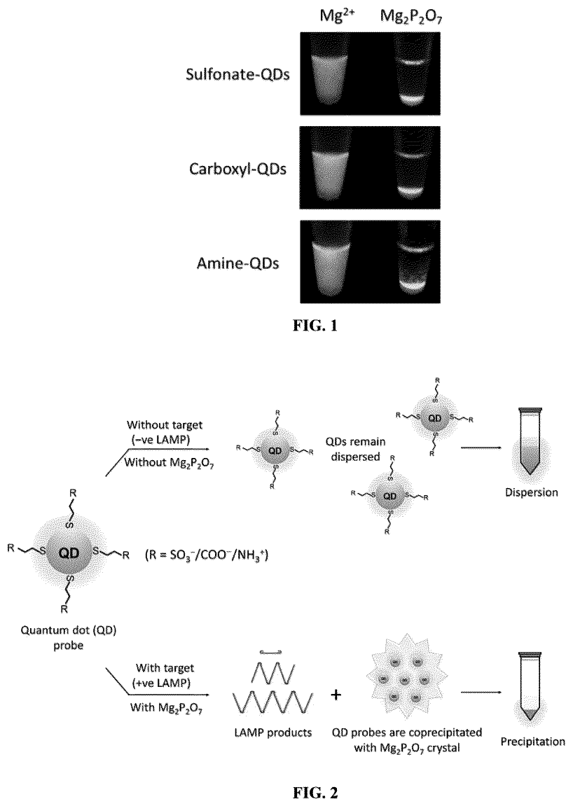

Turning to FIG. 1, dispersion and precipitation behaviors of the three types of QD probes according to certain embodiments are tested under simulated LAMP conditions. In FIG. 1, fluorescence images of simulated negative LAMP samples (“Mg²⁺”; comprising 1×isothermal amplification buffer, 2 mM MgCl₂, and QDs) and simulated positive LAMP samples (“Mg₂P₂O₇”; comprising 1×isothermal amplification buffer, 2 mM MgCl₂, 1.4 mM K₄P₂O₇, and QDs) were taken under 365 nm ultraviolet excitation. The 1×isothermal amplification buffer contained 20 mM Tris-HCl, 10 mM (NH₄)₂SO₄, 50 mM KCl, 2 mM MgSO₄, and 0.1% Tween 20 (pH 8.8). The samples were incubated at 65° C. for 1 h. For a positive LAMP sample (presence of target sequence), in addition to target sequence amplification, P₂O₇⁴⁻ ions are produced, which complex with Mg²⁺ ions (present in nucleic acid amplification reactions as enzyme cofactor) to form insoluble Mg₂P₂O₇ crystals. The size of each individual Mg₂P₂O₇ crystal was ˜0.5-0.8 μm. These crystals are likely spherical clusters of thin and plate-like structures.

From the results in FIG. 1, sulfonate-, carboxyl-, and amine-QDs all remained dispersed in a simulated negative LAMP sample (without Mg₂P₂O₇ crystals; with Mg²⁺ but without P₂O₇⁴⁻; uniform green fluorescence throughout the solution under ultraviolet illumination). On the other hand, sulfonate-, carboxyl-, and amine-QDs all became precipitated in a simulated positive LAMP sample (with Mg₂P₂O₇ crystals; green fluorescent precipitates). For sulfonate- and carboxyl-QDs, in the negative sample, (tris(hydroxymethyl)aminomethane and Tween 20 stabilized the particles against complexation-induced aggregation (between Mg²⁺ and sulfonate/carboxyl), whereas in the positive sample, the particles were embedded within Mg₂P₂O₇ crystals via complexation of Mg²⁺ with P₂O₇⁴⁻ and sulfonate/carboxyl and thus coprecipitation occurred. For amine-QDs, in the negative sample, electrostatic repulsion (positively charged amine-QDs) rendered the particles dispersed, whereas in the positive sample, the particles were embedded within Mg₂P₂O₇ crystals via electrostatic attraction and hydrogen bonding (positively charged amine and negatively charged P₂O₇⁴⁻) and thus coprecipitation occurred. Notably, amine-QDs became negatively charged in the presence of P₂O₇⁴⁻ (amine-QDs: +4.9 mV; amine-QDs with P₂O₇⁴⁻: −22.4 mV), providing evidence for the strong binding between amine-QDs and P₂O₇⁴⁻.

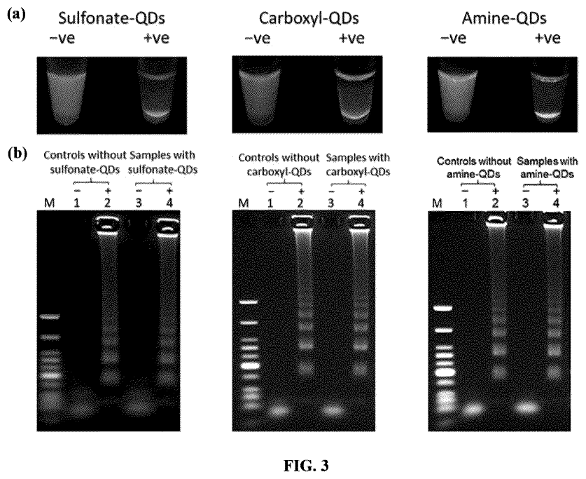

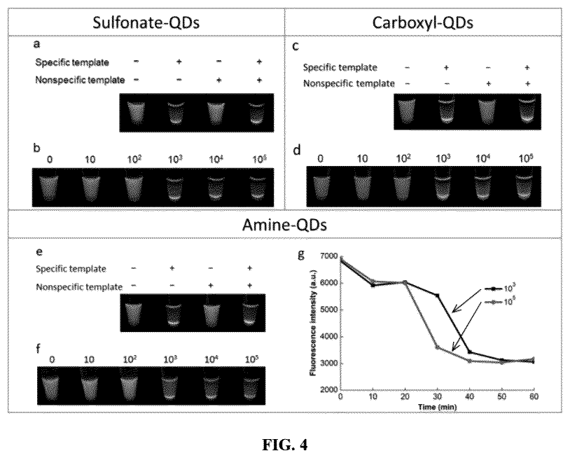

As schematically depicted in FIG. 2, in a negative LAMP sample (absence of specific nucleic acid sequence), QD probes remain dispersed and the mixture appears uniform green fluorescence under UV illumination. On the other hand, in a positive LAMP sample (presence of specific nucleic acid sequence), QD probes are coprecipitated with Mg₂P₂O₇ crystals and a green fluorescent precipitate is resulted. Therefore, the three types of modified QDs according to certain embodiments of the present invention were tested in a closed-tube LAMP assay on samples with or without the target nucleic acid sequence to be amplified. Reaction mixtures comprised one type of QD probes and other standard LAMP reagents. Lambda DNA was used as the specific template. As shown in FIG. 3a, after incubation at 65° C. for 1 h, the corresponding QD probes in the negative LAMP samples (“−ve”; without lambda DNA) remained dispersed, whereas those in the positive LAMP samples (“+ve”; with 10⁵ copies of lambda DNA; 8 fM in a reaction volume of 20 L) were precipitated. Controls without the corresponding QDs were included for comparison. The corresponding agarose gel electrophoresis results shown in FIG. 3b confirmed that successful amplification was achieved in the positive LAMP samples with characteristic ladder-like products (lanes 2 and 4), indicating good compatibility of all three QD probes with LAMP.

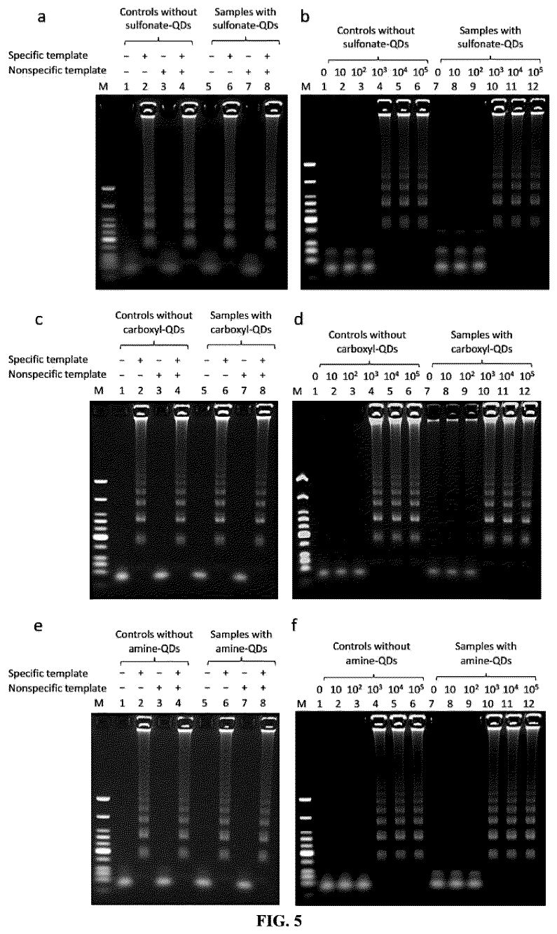

Turning to FIG. 4, detection performances in terms of specificity and sensitivity of the corresponding modified QDs according to certain embodiments are shown by incubating different modified QDs with different combinations of samples, in which lambda DNA and pBR322 DNA were employed as the specific and nonspecific templates, respectively.

From the specificity perspective, FIGS. 4a, 4c, and 4e show that after incubation at 65° C. for 1 h, upon 365 nm ultraviolet excitation to the corresponding modified QDs, the samples containing the nonspecific template only (10⁵ copies) appeared as green dispersion, similar to the samples without any template. On the other hand, green precipitates were detected in the samples containing the specific template only and those containing both the specific and nonspecific templates.

To test the sensitivity of different modified QDs, different copy numbers of lambda DNA (0, 10¹, 10², 10³, 10⁴, and 10⁵) were employed. The results indicated that the samples with 10² copies or less of lambda DNA remained dispersed while green precipitates were detected in the samples with 10³ copies or more (FIGS. 4b, 4d, and 4f).

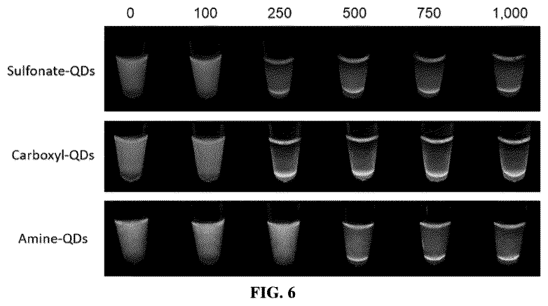

The specificity and sensitivity of the three different modified QDs according to certain embodiments of the present invention in the closed-tube LAMP assay are further demonstrated by agarose gel electrophoresis, the results of which are shown in FIG. 5. As seen in FIG. 5, characteristic ladder-like LAMP products could only be observed in the samples with lambda DNA (specific template) in the specificity tests and the samples containing 10³ copies or more of the lambda DNA in the sensitivity tests, confirming that a successful LAMP was achieved in these samples. In particular, the precipitation process of amine-QDs in the samples with 10³ or 10⁵ copies of lambda DNA was monitored by measuring the fluorescence intensity at 530 nm of the supernatant at 10 min intervals (excited at 360 nm; the samples were briefly centrifuged prior to the supernatant sampling/pipetting), and the change in fluorescence intensity in different set of samples was plotted and shown in FIG. 4g. For the samples with 10⁵ copies of lambda DNA, the fluorescence intensity decreased sharply from 20 min to 30 min, while for the sample with 10³ copies, the fluorescence intensity decreased sharply from 30 min to 40 min. These results suggest the feasibility of the modified QDs in the present invention for real-time monitoring and quantitative analysis.

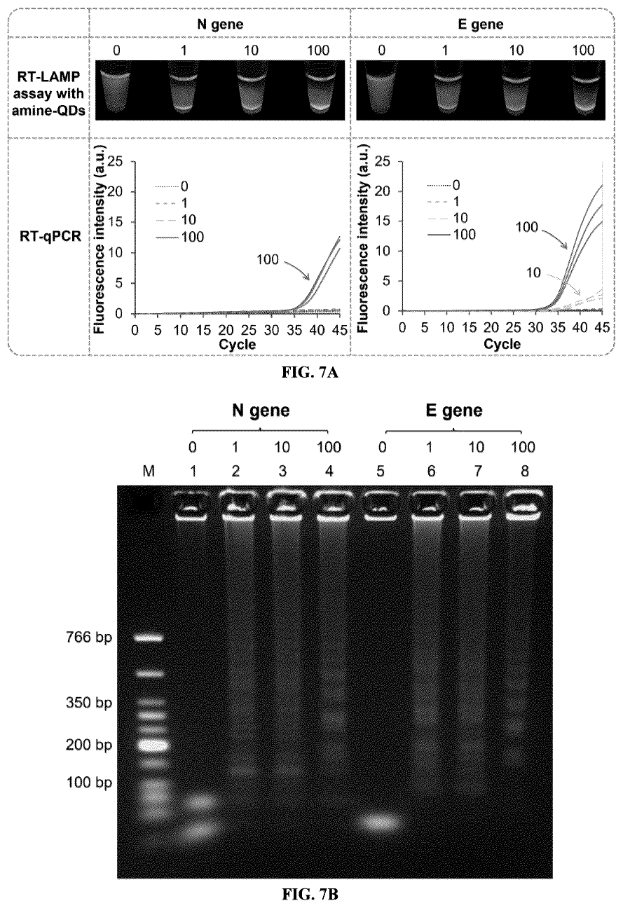

Turning to FIG. 6, the limit of detection (LOD) for three different modified QDs according to certain embodiments of the present invention in LAMP assay is determined. Various copy numbers of lambda DNA (0, 100, 250, 500, 750, 1,000) were used. As seen in FIG. 6, the LODs of the LAMP assays with sulfonate-, carboxyl-, and amine-modified QDs were 250, 250, and 500 copies, respectively.

The following examples and the accompanied drawings are intended to assist the understanding of various embodiments of the present invention, and should not be considered limiting the scope of the invention. The scope of the invention should be referred to the appended claims.

### EXAMPLES

### Example 1—Preparation of Sulfonate-, Amine-, and Carboxyl-Modified Quantum Dot Probes

The preparation of sulfonate-QDs and amine-QDs was based on a reported ligand exchange method with modifications [Lee et al., Nanotechnology, 21, 285102 (2010)]. Briefly, 200 μL of 1 mg/mL oleic acid-capped CdSeS/ZnS QDs (diameter of 6 nm; emission peak at 540 nm; dispersed in toluene; Sigma-Aldrich) were added with 200 μL of chloroform, 200 μL of methanol, and 2 mL of acetone, successively. The above mixture was then centrifuged at 10 krpm for 20 min (Centrifuge 5415D, Eppendorf). After removing the supernatant, the precipitate was dissolved in 200 μL of hexane and added with 2 mL of acetone, followed by another centrifugation at 10 krpm for 20 min. The obtained precipitates were dried at room temperature for 1 h. The dried precipitates were then dissolved in a small amount of chloroform and mixed with 0.5-1 mL 50 mg/mL cysteamine hydrochloride solution (for amine-QDs) or sodium 2-mercaptoethanesulfonate solution (for sulfonate-QDs). Subsequently, the two-layered mixture was sonicated (WiseClean WUC-A011H ultrasonic cleaner, Daihan Scientific).

The top layer containing functionalized QDs was then collected and purified with Amicon ultracentrifugal filters (Millipore) by three cycles of dilution/concentration through ultracentrifugation. The as-prepared amine-QDs and sulfonate-QDs were stored at 4° C. until use. The extinction coefficient of the oleic acid-capped CdSeS/ZnS QDs provided by the manufacturer was used for concentration determination of sulfonate-QDs and amine-QDs by the Beer-Lambert law. For carboxyl-QDs, the mercaptoacetic acid-modified CdSe/ZnS QDs (10 mg/mL; emission in green; Xi'an Ruixi Biological Technology) were also purified through ultracentrifugation and stored at 4° C. until use.

### Example 2—Simulated Precipitation Test with Magnesium Pyrophosphate Crystals

To investigate the behaviors of the three types of modified QD probes in simulated positive LAMP samples (“Mg₂P₂O₇”), a mixture (20 L) containing 1×isothermal amplification buffer (20 mM Tris-HCl, 10 mM (NH₄)₂SO₄, 50 mM KCl, 2 mM MgSO₄, 0.1% Tween 20, pH 8.8; New England Biolabs), 2 mM MgCl₂, 60 nM sulfonate-/amine-QDs or 7.5 g/mL carboxyl-QDs, and 1.4 mM K₄P₂O₇ was prepared, followed by incubation at 65° C. for 1 h (GeneAmp PCR system 9700, Applied Biosystems). Simulated negative LAMP samples without K₄P₂O₇ (“Mg²⁺”) were included as controls. Fluorescence readout was achieved with 365 nm UV excitation (EN-280L/FE, Spectroline, USA). These results are shown in FIG. 1A.

### Example 3—LAMP Coupled with QD Probes for Lambda DNA Detection

A closed-tube LAMP detection scheme based on the three types of QD probes is illustrated in FIG. 2. Six LAMP primers (SEQ ID NOs: 1-6; Table 1) were utilized for amplifying lambda DNA (HPLC-purified; Integrated DNA Technologies) (Notomi et al., Nucleic Acids Res., 28, e63 (2000)). A reaction mixture (20 L) comprising 1×isothermal amplification buffer, deoxyribonucleotide triphosphate (dNTPs; 1.4 mM; 0.35 mM each), lambda-FIP (0.8 μM), lambda-BIP (0.8 μM), lambda-F3 (0.2 μM), lambda-B3 (0.2 μM), lambda-loop F (0.4 μM), lambda-loop B (0.4 μM), betaine (0.2 μM; Sigma-Aldrich), QD probes (40 nM for sulfonate-/amine-QDs or 5 g/mL for carboxyl-QDs), Bst 2.0 DNA polymerase (0.32 units/μL; New England Biolabs), and lambda DNA (10⁵ copies for a positive sample “+ve” and 0 copies for a negative sample “−ve”) was incubated at 65° C. for 1 h (GeneAmp PCR system 9700). Parallel reactions were performed without QD probes. After LAMP reaction, the samples were observed with ultraviolet excitation. For agarose gel electrophoresis, LAMP products (8 L) and a low molecular weight DNA ladder (New England Biolabs) were stained with 1 μL of 100×SYBR Green I nucleic acid gel stain (Thermo Fisher Scientific) in darkness at room temperature for 15 min. Mixtures of the stained LAMP products and orange DNA loading dye (2 μL; Thermo Fisher Scientific), together with the stained DNA ladder, were then loaded into an agarose gel (2 wt % in 0.5×TBE buffer: 45 mM Tris, 45 mM boric acid, and 1 mM EDTA; pH 8.0) and subjected to electrophoresis at 100 V for 45-60 min. The gel was then visualized by UV transillumination (Gel Doc XR+ system, Bio-Rad). These results are shown in FIG. 3.

TABLE 1

| SEQ | | |
|---|---|---|
| ID | | |
| NO | Name | Sequence (5′ to 3′) |
| 1 | lambda-FIP | CAGCCAGCCGCAGCACGTTCGCTCATAGGAGAT |
| | | ATGGTAGAGCCGC |
| 2 | lambda-BIP | GAGAGAATTTGTACCACCTCCCACCGGGCACAT |
| | | AGCAGTCCTAGGGACAGT |
| 3 | lambda-F3 | GGCTTGGCTCTGCTAACACGTT |
| 4 | lambda-B3 | GGACGTTTGTAATGTCCGCTCC |
| 5 | lambda-LF | CTGCATACGACGTGTCT |
| 6 | lambda-LB | ACCATCTATGACTGTACGCC |
| 7 | SARS-CoV-2-N-FIP | TCTGGCCCAGTTCCTAGGTAGTCCAGACGAATTC |
| | | GTGGTGG |
| 8 | SARS-CoV-2-N-BIP | AGACGGCATCATATGGGTTGCACGGGTGCCAAT |
| | | GTGATCT |
| 9 | SARS-CoV-2-N-F3 | TGGCTACTACCGAAGAGCT |
| 10 | SARS-CoV-2-N-B3 | TGCAGCATTGTTAGCAGGAT |
| 11 | SARS-CoV-2-N-LF | GGACTGAGATCTTTCATTTTACCGT |
| 12 | SARS-CoV-2-N-LB | ACTGAGGGAGCCTTGAATACA |
| 13 | SARS-CoV-2-E-FIP | ACCACGAAAGCAAGAAAAAGAAGTTCGTTTCGG |
| | | AAGAGACAG |
| 14 | SARS-CoV-2-E-BIP | TTGCTAGTTACACTAGCCATCCTTAGGTTTTACA |
| | | AGACTCACGT |
| 15 | SARS-CoV-2-E-F3 | TGAGTACGAACTTATGTACTCAT |
| 16 | SARS-CoV-2-E-B3 | TTCAGATTTTTAACACGAGAGT |
| 17 | SARS-CoV-2-E-LF | CGCTATTAACTATTAACG |
| 18 | SARS-CoV-2-E-LB | GCGCTTCGATTGTGTGCGT |
| 19 | SARS-CoV-2-N-F | CACATTGGCACCCGCAATC |
| 20 | SARS-CoV-2-N-R | GAGGAACGAGAAGAGGCTTG |
| 21 | SARS-CoV-2-N-P | FAM-ACTTCCTCAAGGAACAACATTGCCA-BHQ1 |
| 22 | SARS-CoV-2-E-F | ACAGGTACGTTAATAGTTAATAGCGT |
| 23 | SARS-CoV-2-E-R | ATATTGCAGCAGTACGCACACA |
| 24 | SARS-CoV-2-E-P | FAM-ACACTAGCCATCCTTACTGCGCTTCG- |
| | | ZEN/IBFQ |
| 25 | AIV-H9-FIP | GCCCCACATGAAAAGAATGTTCTTTGTTGACTCA |
| | | AAAGAACAACGC |
| 26 | AIV-H9-BIP | AATCACCCACCCACTGATACTATCTTCTGTTGCC |
| | | ACACTTG |
| 27 | AIV-H9-F3 | CATTCTACAGAAGCATGAGATG |
| 28 | AIV-H9-B3 | AATGGTTTGAAGGCCCTAT |
| 29 | AIV-H9-LF | GTGTATTGGGCGTCTTGAATAGG |
| 30 | AIV-H9-LB | ACGCAGACAGATCTGTACACA |
| 31 | AIV-H9-F | ATTTATTCGACTG(C/T)CGCC |
| 32 | AIV-H9-R | ATGTTGCA(C/T)CTGCAAGA |
| 33 | AIV-H9-P | FAM-TGC(A/G)AT(A/G)GGGTTTGCGCC-BHQ1 |

### Example 4—Specificity and Sensitivity Evaluation of QD Probes in LAMP Assay

For specificity test, four different template combinations with or without lambda DNA (specific template) and pBR322 DNA (nonspecific template) were employed. In addition to fluorescence readout, the LAMP reaction products were further analyzed by agarose gel electrophoresis. For sensitivity test, different copy numbers of the lambda DNA (0, 10, 10², 10³, 10⁴, and 10⁵) were utilized. The amplification results of the samples in both specificity and sensitivity tests were analyzed by fluorescence readout (FIG. 4a-f) and agarose gel electrophoresis (FIG. 5). For amine-QDs, fluorescence measurement of the samples containing 10³ and 10⁵ copies of lambda DNA was performed at 10 min intervals. The samples were centrifuged at 2 krpm for 30 s, and the supermatants (15 μL) were collected and added to 384-well black polystyrene microplates (Greiner). The fluorescence intensity at 530 nm was measured under 360 nm excitation (Synergy HTX Multi-Mode Reader, BioTek) and the results are shown in FIG. 4g. Moreover, various copy numbers of the lambda DNA template (0-1,000) were tested for limit of detection (LOD) determination of the LAMP assays with the three types of QD probes. The results were checked by fluorescence readout (FIG. 6).

### Example 5—RT-LAMP Coupled with QD Probes

Reverse transcription-LAMP (RT-LAMP) can be performed under similar conditions as LAMP, except with the addition of 0.2-0.5 units/μL WarmStart reverse transcriptase (New England Biolabs), 6 mM MgSO₄, and an RNA template (instead of a DNA template). The samples can be incubated at 65° C. for 30-60 min. Amplification detection is achieved based on the dispersion/precipitation behavior of the QD probes after the incubation or real-time fluorescence monitoring during the incubation.

### Example 6—RT-LAMP Coupled with Amine-QDs for SARS-CoV-2 RNA Detection

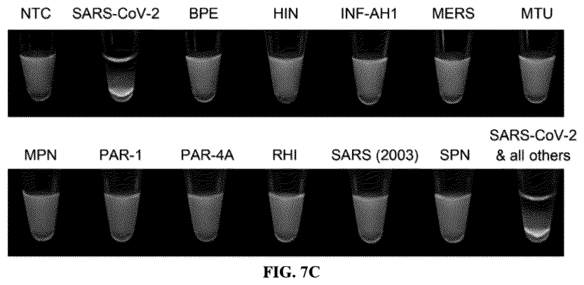

RNA of severe acute respiratory syndrome coronavirus 2 (SARS-CoV-2), the causative agent of coronavirus disease 2019 (COVID-19), was chosen to demonstrate the applicability of amine-QDs for the one-step RT-LAMP assay. Six RT-LAMP primers for amplifying SARS-CoV-2 nucleocapsid (N) gene (SEQ ID NOs: 7-12) and six RT-LAMP primers for amplifying SARS-CoV-2 envelope (E) gene (SEQ ID Nos: 13-18) were used (Zhang et al. Enhancing Colorimetric Loop-Mediated Isothermal Amplification Speed and Sensitivity with Guanidine Chloride. BioTechniques 2020, 69, 178-185). FIG. 7A shows the sensitivity tests targeting the N gene and the E gene in terms of fluorescence images (top row) taken under UV excitation (˜360 nm). After incubation at 65° C. for 40 min, for both the N and E genes, all the positive samples (1, 10, and 100 copies; partial precipitation of amine-QDs, obvious green fluorescent precipitate) were clearly distinguishable from the negative sample (0 copies; well dispersion of amine-QDs, uniform green fluorescence). The fluorescence readout results were confirmed by agarose gel electrophoresis analysis (FIG. 7B). The single-copy detection capability of this amine-QD-based one-step RT-LAMP compared favorably with RT-qPCR (the plots of fluorescence intensity vs cycle number for different copy numbers from 0 to 100 (triplicated for each) in the bottom row of FIG. 7A, where for the E gene, 10 copies could be detected but not 1 copy; for the N gene, 100 copies could be detected but not 10 copies). Each of the N and E genes of SARS-CoV-2 RNA were amplified by two RT-qPCR primers and one TaqMan probe, represented by SEQ ID NOs: 19-21 and 22-24, respectively (Corman et al. Detection of 2019 Novel Coronavirus (2019-nCoV) by Real-Time RT-PCR. Eurosurveillance 2020, 25, 2000045). For RT-qPCR assays, samples (20 L) comprised 1×QuantiFast Pathogen Master Mix, 1× QuantiFast Pathogen RT Mix, 1×Internal Control Assay, 1× Internal Control RNA, SARSCoV-2-N/E-F (N: 0.6 μM; E: 0.4 μM), SARS-CoV-2-N/E-R (N: 0.8 μM; E: 0.4 μM), SARS-CoV-2-N/E-P (N: 0.2 μM; E: 0.2 μM), and SARS-CoV-2 RNA (0-100 copies). The temperature profile involved RT at 50° C. for 20 min and initial denaturation at 95° C. for 5 min followed by 45 thermal cycles of denaturation at 95° C. for 15 s and annealing/extension at 60° C. for 30 s. All RT-qPCR assays were performed in triplicate.

For the specificity test with RT-LAMP, different templates were tested, including no template control, SARS-CoV-2 (specific template; 10² copies), and 11 nonspecific templates (10³ copies; Bordetella pertussis DNA (BPE), Haemophilus influenzae DNA (HIN), influenza A H1 RNA (INF-AH1), Middle East respiratory syndrome coronavirus RNA (MERS), Mycobacterium tuberculosis DNA (MTU), Mycoplasma pneumoniae DNA (MPN), parainfluenza 1 RNA (PAR-1), parainfluenza 4A RNA (PAR-4A), rhinovirus RNA (RH1), SARS (2003) coronavirus RNA (SARS (2003)), and Streptococcus pneumoniae DNA (SPN)). After incubation at 65° C. for 35 min, the samples were visualized under ultraviolet excitation. As shown in FIG. 7C, after incubation at 65° C. for 35 min, green fluorescent precipitates only occurred in the samples containing the specific template (SARS-CoV-2 RNA; alone or in the presence of all other 11 nonspecific templates), demonstrating the excellent specificity of the amine-QD-based one-step RT-LAMP assay.

### Example 7—RT-LAMP Coupled with Amine-QDs for Avian Influenza Virus RNA Detection from Live Chicken Swab

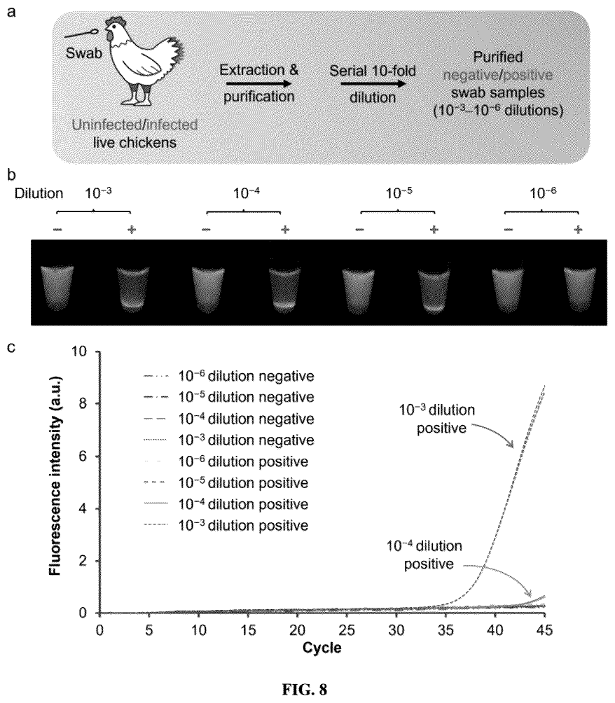

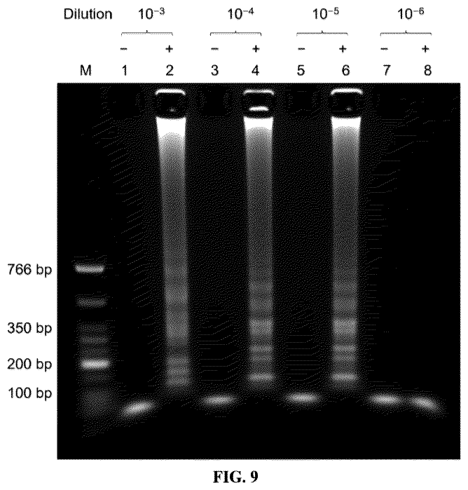

To evaluate the performance of the amine-QD-based one-step RT-LAMP assay for real application, oropharyngeal/cloacal swab samples from live chickens were tested for avian influenza virus (AIV) H9 subtype RNA. The samples were first processed to prepare purified RNA extracts. Then, different dilutions of the purified RNA extracts were prepared (10³-fold dilution “10⁻³” and 10-fold serial dilutions down to “10⁻⁶”; FIG. 8a). FIG. 8b shows the fluorescence readout results of two swab samples after RT-LAMP (RT-LAMP samples (20 L) comprised 1×isothermal amplification buffer, six RT-LAMP primers for amplifying AIV H9 subtype RNA (SEQ ID NOs: 25-30): AIV-H9-FIP (1.6 μM), AIV-H9-BIP (1.6 μM), AIV-H9-F3 (0.2 μM), AIV-H9-B3 (0.2 μM), AIV-H9-LF (0.8 μM), AIV-H9-LB (0.8 μM), amine-QDs (40 nM), AMV reverse transcriptase (0.025 units/μL), betaine (0.2 μM), Bst 2.0 DNA polymerase (0.4 units/μL), dNTPs (1.4 mM; 0.35 mM each), and purified RNA extract (1 μL of different dilutions; 10⁻³-10⁻⁶). After incubation at 65° C. for 40 min, the samples were visualized under ultraviolet excitation (365 nm). Amine-QDs of one sample were precipitated for 10⁻³, 10⁴, and 10⁻⁵ dilutions but were dispersed for 10⁻⁶ dilution, indicative of a positive sample (presence of AIV H9 RNA) reaching a single-copy level at 10⁻⁵ dilution. On the other hand, amine-QDs of another sample were dispersed for all the dilutions, indicative of a negative sample (absence of AIV H9 RNA). The amplification results were verified by agarose gel electrophoresis analysis (same protocol as that for lambda DNA) (FIG. 9).

RT-qPCR was also performed for further verification of the two swab samples (samples (20 L) comprised 1×QuantiFast Pathogen Master Mix, 1×QuantiFast Pathogen RT Mix, 1×Internal Control Assay, 1×Internal Control RNA, two RT-qPCR primers and one TaqMan probe (SEQ ID NOs: 31-33): AIV-H9-F (0.4 μM), AIV-H9-R (0.4 μM), AIV-H9-P (0.2 μM), and purified RNA extract (1 μL of different dilutions; 10⁻³-10⁻⁶). The temperature profile involved RT at 50° C. for 20 min initial denaturation at 95° C. for 5 min followed by 45 thermal cycles of denaturation at 95° C. for 15 s and annealing/extension at 60° C. for 30 s. All RT-qPCR assays were performed in duplicate). The results confirmed that one sample was positive and another sample was negative (FIG. 8c), consistent with the amine-QD-based one-step RT-LAMP results. It should be noted that, with RT-qPCR, the positive sample was detectable down to 10⁻⁴ dilution only (versus 10⁻⁵ dilution with amine-QD-based one-step RTLAMP). In fact, such 10-fold improvement in the assay sensitivity was also observed with lambda DNA and SARS-CoV-2 RNA.

Although the invention has been described in terms of certain embodiments, other embodiments apparent to those of ordinary skill in the art are also within the scope of this invention. Accordingly, the scope of the invention is intended to be defined only by the claims which follow.

### INDUSTRIAL APPLICABILITY

The present invention provides a first-of-its-kind one-step isothermal nucleic acid amplification platform and detection method coupled with quantum dot fluorescent reporter in a closed-tube manner. Apart from being universal, closed-tube format (no carryover contamination), clear and easy result readout, low-cost and simple preparation, and isothermal (simple temperature control), this assay platform is highly specific and sensitive (inherent advantage of LAMP). With easy reagent transportation and storage (lyophilized LAMP reagents) and high robustness (high tolerance to unpurified samples) of LAMP, the present invention is easily applicable to point-of-care and on-site nucleic acid testing. The assay platform exhibits great potential for further applications in clinical diagnosis, food safety monitoring, and environment surveillance.

What is claimed is:

1. A universal fluorescent probing system for one-step isothermal nucleic acid amplification assay, comprising a plurality of functionalized fluorescent nanoprobes capable of forming co-precipitates in the presence of positive isothermal amplification reaction products along with a target sequence amplification.

2. The system of claim 1, wherein the plurality of functionalized fluorescent nanoprobes comprises one or more semiconductor quantum dots (QDs) with one or more surface modifications.

3. The system of claim 2, wherein the one or more semiconductor QDs comprise cadmium selenide sulfide/zinc sulfide (CdSeS/ZnS core/shell) quantum dots.

4. The system of claim 2, wherein the one or more semiconductor QDs are surface modified by one of 2-mercaptoethanesulfonate, mercaptoacetic acid, and cysteamine to result in sulfonate-QDs, carboxyl-QDs, and amine-QDs, respectively.

5. The system of claim 4, wherein the positive isothermal amplification reaction products with which the plurality of functionalized fluorescent nanoprobes is capable of forming coprecipitates include magnesium pyrophosphate (Mg₂P₂O₇) crystals.

6. The system of claim 5, wherein the sulfonate-QDs or carboxyl-QDs form coprecipitates via complexation between Mg²⁺ and P₂O₇⁴⁻ of the Mg₂P₂O₇ crystals with the sulfonate or carboxyl group of the sulfonate-QDs and carboxyl-QDs in the course of the isothermal amplification reaction.

7. The system of claim 4, wherein the amine-QDs form coprecipitates via electrostatic attraction between P₂O₇⁴⁻ of the Mg₂P₂O₇ crystals and amine group of the amine-QDs in the course of the isothermal amplification reaction.

8. The system of claim 1, wherein the isothermal amplification reaction comprises loop-mediated isothermal amplification (LAMP) and reverse transcription-loop-mediated isothermal amplification (RT-LAMP) reactions.

9. The system of claim 8, wherein the LAMP reaction is performed at about 65° C. for 1 hour or less.

10. A method for closed-tube detection of a target nucleic acid sequence in a sample based on an isothermal amplification reaction, the method comprising:
providing the plurality of functionalized fluorescent nanoprobes of the universal fluorescence probing system of claim 1 to an isothermal amplification reaction mixture containing the sample;
incubating the plurality of the functionalized fluorescent nanoprobes with the isothermal amplification reaction mixture containing the sample at an isothermal amplification temperature for a time interval;
analyzing the isothermal amplification reaction mixture during or after said incubating by a fluorescence reader, wherein supernatant portion of the reaction mixture is analyzed; and
determining the presence of the target sequence in the sample by detecting any fluorescence precipitates formed in the isothermal amplification reaction mixture during or after said incubating;
otherwise, detecting a fluorescence dispersion in the isothermal amplification reaction mixture after said incubating for verifying the absence of the target sequence in the sample.

11. The method of claim 10, wherein the isothermal amplification reaction mixture comprises isothermal amplification reagents capable of forming Mg₂P₂O₇ crystals when positive amplicons of the target sequence are generated by either LAMP or RT-LAMP reaction.

12. The method of claim 10, wherein an analyte of the sample comprises one or more types of nucleic acids directly obtained or isolated from a test subject, object, or biological sample.

13. The method of claim 10, wherein the isothermal amplification temperature is about 65° C., and the time interval for said isothermal amplification is about 1 hour or less.

14. The method of claim 11, wherein the LAMP or RT-LAMP reaction is carried out in any device with uniform heating and cooling capabilities and a time control mechanism.

15. A method of fabricating the universal fluorescence probing system of claim 1 for an isothermal amplification reaction, the method comprising:
selecting a semiconductor quantum dot;
modifying surface of said semiconductor quantum dot with one or more functional groups such that the one or more functional groups are capable of interacting with Mg²⁺ and/or P₂O₇⁴⁻ to form coprecipitates.

16. A kit for detecting a target nucleic acid sequence in a sample, the kit comprising any one or more of the functionalized fluorescent nanoprobes of the universal fluorescence probing system according to claim 1, reaction components for LAMP or RT-LAMP reaction including isothermal amplification buffer, deoxynucleotides, enzymes, betaine, LAMP primers with respect to the target sequence, stabilizers or additives for facilitating formation of the coprecipitates, a control sample, and solvent or solution for dissolving any of the reaction components.
